(19) United States
(12) Patent Application Publication
(10) Pub. No.: US 2025/0134388 A1
(43) Pub. Date: May 1, 2025

(54) POINT-OF-CARE TRANSESOPHAGEAL ECHO-OXIMETER WITH A MINIATURE NASAL PROBE FOR CENTRAL HEMODYNAMICS ASSESSMENT

(71) Applicant: The General Hospital Corporation, Boston, MA (US)

(72) Inventors: Li Li, Quincy, MA (US); Guillermo J. Tearney, Cambridge, MA (US)

(73) Assignee: The General Hospital Corporation, Boston, MA (US)

(21) Appl. No.: 18/682,953

(22) PCT Filed: Aug. 10, 2022
(86) PCT No.: PCT/US2022/074744

§ 371 Date: Feb. 12, 2024

Related U.S. Application Data
(60) Provisional application No. 63/231,607, filed on Aug. 10, 2021.

(51) Int. Cl. A61B 5/00; A61B 5/145

(52) U.S. Cl. CPC A61B 5/0095; A61B 5/14542; A61B 5/6853

(57) ABSTRACT

An apparatus for transesophageal echo-oximetry, including: an insertion tube having an optical fiber disposed therein: a probe disposed at an end of the insertion tube, the probe including: an acoustic transducer, and a reflector aligned with the acoustic transducer and the optical fiber: a controller in communication with the acoustic transducer. the controller configured to: generate an ultrasonic image of a sample using the acoustic transducer and the reflector, direct light from the optical fiber toward the reflector and into the sample, collect photoacoustic signals from the sample based on the directed light using the acoustic transducer and the reflector, and determine a blood oxygenation level in the sample based on the photoacoustic signals.

### CROSS-REFERENCE TO RELATED APPLICATIONS

The present application is based on and claims priority from U.S. Patent Application Ser. No. 63/231,607, filed on Aug. 10, 2021, the entire disclosure of which is incorporated herein by reference.

### STATEMENT REGARDING FEDERALLY SPONSORED RESEARCH

This invention was made with government support under grant number HU0001-17-2-0009 awarded by the Uniform Services University of the Health Sciences. The government has certain rights in the invention.

### BACKGROUND

Real-time assessment of central hemodynamics at the point of care could advance the treatment of cardiovascular, trauma, and intensive care patients. The standard approach which uses highly invasive pulmonary catheters provides comprehensive assessment of hemodynamic functions but is not practical at the point of care. Echocardiography is gaining popularity as a noninvasive alternative yet falls short of answering key questions about parameters such as the body's oxygen adequacy.

### SUMMARY OF THE INVENTION

Accordingly, new systems, methods, and apparatus for transesophageal echo-oximetry are desirable.

Hemorrhagic shock is a preventable but sometimes fatal condition. Mixed venous oxygenation (SvO₂), measured from the pulmonary artery, is a sensitive, immediate, and reliable indicator of blood loss and circulatory shock. In current clinical practice, SvO₂ measurement requires placing pulmonary artery catheters (PAC) through a highly invasive procedure associated with a significant risk of severe complications. This procedure requires a sterilized environment and general anesthesia, making it impractical to be performed at the point of care. Embodiments of the TEO disclosed herein are capable of continuous monitoring of SvO₂ using photoacoustics. Oxygenated and de-oxygenated blood absorb different amounts of light at different wavelengths and emits ultrasound of varying amplitudes, allowing for evaluation of blood oxygenation, while at the same time the TEO device can obtain real-time ultrasonic images of cardiac structures. Combining ultrasound structural and photoacoustic information makes it feasible to derive a comprehensive set of hemodynamic information, such as heart rate, blood flow, blood pressure, preload, afterload, cardiac output, arterial oxygenation, venous oxygenation, oxygen delivery, or oxygen consumption. The TEO technology disclosed herein does not require the placement of a central line and can be used in patients who are not sedated.

Thus, in one embodiment, the disclosure provides an apparatus for transesophageal echo-oximetry, including: an insertion tube having an optical fiber disposed therein; a probe disposed at an end of the insertion tube, the probe including: an acoustic transducer, and a reflector aligned with the acoustic transducer and the optical fiber. The apparatus may also include a controller in communication with the acoustic transducer, where the controller may be configured to: generate an ultrasonic image of a sample using the acoustic transducer and the reflector, direct light from the optical fiber toward the reflector and into the sample, collect photoacoustic signals from the sample based on the directed light using the acoustic transducer and the reflector, and determine a blood oxygenation level in the sample based on the photoacoustic signals.

In certain embodiments of the apparatus, the probe may further include a micromotor coupled to the reflector and in communication with the controller and the controller may be further configured to: rotate the reflector using the micromotor, generate the ultrasonic image of the sample using the acoustic transducer and the rotating reflector, direct light from the optical fiber toward the rotating reflector and into the sample, collect photoacoustic signals from the sample based on the directed light using the acoustic transducer and the rotating reflector, and determine the blood oxygenation level in the sample based on the photoacoustic signals collected using the rotating reflector.

Some embodiments of the apparatus may further include a pulsed light source coupled to the optical fiber and in communication with the controller. In certain embodiments of the apparatus, the pulsed light source may emit light pulses having a pulse duration of at least 1 ns and no more than 100 ns. In particular embodiments of the apparatus, the pulsed light source may emit light pulses including far-red or near-infrared light (600˜2500 nm). In various embodiments of the apparatus, the pulsed light source may be switchable between two different wavelengths of light. In some embodiments of the apparatus, the pulsed light source may emit light pulses including a wavelength in a range of at least one of 750 nm to 770 nm or 900 nm to 1100 nm. In certain embodiments of the apparatus, the pulsed light source may emit light pulses including a wavelength of at least one of 760 nm or 1053 nm. In other embodiments of the apparatus, the pulsed light source may emit light pulses including a wavelength of at least one of 760 nm or 1064 nm.

In various embodiments of the apparatus, the pulsed light source may emit light pulses having an energy of greater than −10 mJ. In some embodiments of the apparatus, the pulsed light source may be air cooled. In particular embodiments of the apparatus, the pulsed light source may include a power supply including a battery. In various embodiments of the apparatus, the pulsed light source may be coupled to the optical fiber using a microlens array and a spherical lens to project an output of the pulsed light source onto an end of the optical fiber.

In certain embodiments of the apparatus, the pulsed light source may include: a first resonant cavity powered by a pair of laser diodes; a second resonant cavity in optical communication with the first optical cavity; an electro-optic modulator; and an output configured to emit light pulses. The electro-optic modulator may have a first position which transmits light from the first resonant cavity to the output, the electro-optic modulator may have a second position which transmits light from the first resonant cavity to the second resonant cavity, the light pulses may be emitted from the output including a first wavelength from the first resonant cavity when the electro-optic modulator is in the first position, and the light pulses may be emitted from the output including a second wavelength different from the first wavelength from the second resonant cavity when the electro-optic modulator is in the second position.

In some embodiments of the apparatus, the optical fiber may extend through an opening in the acoustic transducer. In certain embodiments of the apparatus, the probe may include a housing having the reflector and the acoustic transducer disposed therein, and the housing may include an acoustic coupling fluid disposed therein.

In certain embodiments of the apparatus, the controller, when determining a blood oxygenation level in the sample based on the photoacoustic signals, may be further configured to: determine a blood oxygenation level in the sample based on the photoacoustic signals once per second. In particular embodiments of the apparatus, the controller, when determining a blood oxygenation level in the sample based on the photoacoustic signals, may be further configured to: determine at least one of heart rate, blood flow, blood pressure, preload, afterload, cardiac output, oxygen delivery, or oxygen consumption in the sample based on the photoacoustic signals and the ultrasonic image of the sample.

In various embodiments of the apparatus, a diameter of the probe may be 6 mm or less. In particular embodiments of the apparatus, the probe may be configured to be delivered through a transnasal tube. In certain embodiments of the apparatus, the probe may be disposed within a balloon, and the balloon may be inflated using a fluid. Some embodiments of the apparatus may further include a portable power supply, and the apparatus may be configured to be stored in a portable case.

Some embodiments of the disclosure provide a method for transesophageal echo-oximetry, including: providing an insertion tube having an optical fiber disposed therein and a probe disposed at an end of the insertion tube, the probe including: an acoustic transducer, and a reflector aligned with the acoustic transducer and the optical fiber. The method may include: generating, using a controller in communication with the acoustic transducer, an ultrasonic image of a sample using the acoustic transducer and the reflector, directing, using the controller, light from the optical fiber toward the reflector and into the sample, collecting, using the controller, photoacoustic signals from the sample based on the directed light using the acoustic transducer and the reflector, and determining, using the controller, a blood oxygenation level in the sample based on the photoacoustic signals.

In certain embodiments of the method, the probe may further include a micromotor coupled to the reflector and in communication with the controller, and the method may further include: rotating the reflector using the micromotor, generating the ultrasonic image of the sample using the acoustic transducer and the rotating reflector, directing light from the optical fiber toward the rotating reflector and into the sample, collecting photoacoustic signals from the sample based on the directed light using the acoustic transducer and the rotating reflector, and determining the blood oxygenation level in the sample based on the photoacoustic signals collected using the rotating reflector.

Some embodiments of the method may further include: providing a pulsed light source coupled to the optical fiber and in communication with the controller. Certain embodiments of the method may further include emitting, using the pulsed light source, light pulses having a pulse duration of at least 1 ns and no more than 100 ns. Particular embodiments of the method may further include emitting, using the pulsed light source, light pulses including far-red or near-infrared light (600˜2500 nm). In various embodiments of the method, emitting light pulses may further include: switching, using the pulsed light source, between two different wavelengths of light. In some embodiments of the method, emitting light pulses may further include: emitting, using the pulsed light source, light pulses including a wavelength in a range of at least one of 750 nm to 770 nm or 900 nm to 1100 nm. In various embodiments of the method, emitting light pulses may further include: emitting, using the pulsed light source, light pulses including a wavelength of at least one of 760 nm or 1053 nm or a wavelength of at least 760 nm or 1064 nm.

In some embodiments of the method, emitting light pulses may further include: emitting, using the pulsed light source, light pulses having an energy of greater than 10 mJ. In certain embodiments of the method, providing a pulsed light source may further include: providing the pulsed light source, wherein the pulsed light source is air cooled. In particular embodiments of the method, providing a pulsed light source may further include: providing the pulsed light source, wherein the pulsed light source includes a power supply including a battery.

Some embodiments of the method may further include coupling the pulsed light source to the optical fiber using a microlens array and a spherical lens and projecting an output of the pulsed light source onto an end of the optical fiber based on coupling the pulsed light source to the optical fiber.

In various embodiments of the method, providing an insertion tube having an optical fiber disposed therein may further include: extending the optical fiber through an opening in the acoustic transducer. In some embodiments of the method, providing an insertion tube having an optical fiber disposed therein and a probe disposed at an end of the insertion tube may further include: providing a housing having the reflector and the acoustic transducer disposed therein, wherein the housing may have an acoustic coupling fluid disposed therein.

In particular embodiments of the method, determining a blood oxygenation level in the sample based on the photoacoustic signals may further include: determining a blood oxygenation level in the sample based on the photoacoustic signals once per second. In some embodiments of the method, determining a blood oxygenation level in the sample based on the photoacoustic signals may further include: determining at least one of heart rate, blood flow, blood pressure, preload, afterload, cardiac output, oxygen delivery, or oxygen consumption in the sample based on the photoacoustic signals and the ultrasonic image of the sample.

In certain embodiments of the method, providing an insertion tube having an optical fiber disposed therein and a probe disposed at an end of the insertion tube may further include: providing a probe having a diameter of 6 mm or less. In some embodiments of the method, providing an insertion tube having an optical fiber disposed therein and a probe disposed at an end of the insertion tube may further include: providing a probe configured to be delivered through a transnasal tube.

In various embodiments of the method, providing an insertion tube having an optical fiber disposed therein and a probe disposed at an end of the insertion tube may further include: providing a probe disposed within a balloon, wherein the balloon is inflated using a fluid. Some embodiments of the method may further include: providing a portable power supply, and storing the insertion tube, the probe, the controller, and the portable power supply in a portable case.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows a portable TEO console for in-vivo applications. (Panel A) Photo of the system. (Panel B) Engineering drawing showing the internal configuration of the system. (Panel C) Function block diagram showing connections among system components.

FIG. 2 shows a compact dual-wavelength light source unit. (Panel A) Engineering drawing of the customized OPO laser. (Panel B) Fast wavelength tuning through electro-optic modulation. M: Mirror (e.g. dichroic mirror); EOM: electrooptical modulator (e.g. Pockels cell); ND: YLF: neodymium-doped yttrium lithium fluoride crystal; OC: output coupler; LD: laser diode; SHG: second harmonic generator; PBS: polarizing beam splitter; BD: beam dumper; BBO: barium borate crystal; W: laser window. (Panel C) Efficient nanosecond light coupling through a single optical fiber using a traditional method (top) and the presently disclosed procedures (bottom), where subpanels (A)-(E) show cross-sectional views of the light beam at the indicated points in the transmission.

FIG. 3 shows a transnasal TEO probe. (Panel A) Photo showing transnasal deployment of the probe in an adult nasogastric training mannequin. (Panel B) Engineering drawings showing the internal configuration of the probe. (Panel C) Photos showing the balloon in the deflated (top, 6 mm dia.) and inflated (bottom, 20 mm dia.) states.

FIG. 4 shows a transnasal TEO probe. (Panel A) Engineering drawings of the probe showing the proximal connectors and the internal configuration of the distal measurement tip (inset). (Panel B) Photos of the TEO probe alongside a commercial nasogastric tube. (Panel C) Transnasal placement of a TEO probe in a nasogastric feeding training mannequin.

FIG. 5 shows data related to monitoring blood oxygenation change in vivo by TEO. (Panel A) A frame of the real-time ultrasound images showing the location of the aorta (yellow asterisk) and the angular position (red line) where photoacoustic measurements were taken. Field of view: 10 cm×10 cm. (Panel B) Change in the fraction of inspired oxygen (FiO₂). (Panel C) Aortic oxygen saturation (SaO₂) change in response to FiO₂modulation. Green line: continuous TEO measurements of blood oxygenation. Red diamonds: Blood oxygenation measured from discrete blood samples by a commercial blood gas analyzer. (Panel D) Comparison of SaO₂ measurements obtained by TEO and the blood gas analyzer, showing an excellent correlation. Solid line: line of best fit through linear regression. Dashed line: 95% confidence bands for the linear regression's slope.

FIG. 6 shows a laser safety study of esophageal tissue exposed to TEO light. (Panel A) Photo showing the exposure region. The central region (dashed black circle and black arrow) is exposed to the full power from OPO laser. The two registration sites (red arrows) were marked by red tissue ink to denote the sites for histological analysis. (Panels B-D) Representative NTBC-stained tissue sections exposed at different exposure durations (1, 10, and 30 mins, respectively) show no tissue damage at the laser exposure sites.

FIG. 7 shows an example of a system for transesophageal echo-oximetry in accordance with some embodiments of the disclosed subject matter.

FIG. 8 shows an example of hardware that can be used to implement a computing device and server in accordance with some embodiments of the disclosed subject matter.

FIG. 9 shows an example of a process for transesophageal echo-oximetry in accordance with some embodiments of the disclosed subject matter.

### DETAILED DESCRIPTION

In accordance with some embodiments of the disclosed subject matter, mechanisms (which can include systems, methods, and apparatus) for transesophageal echo-oximetry are provided.

Thus, disclosed herein are various embodiments of procedures for carrying out transesophageal echo-oximetry (TEO), in which a small probe is placed in the esophagus to simultaneously produce sonographic images and spectroscopic photoacoustic measurements from the heart. The probe can be introduced through the nose into the esophagus to minimize the gag reflex and improve patient tolerance. In various embodiments, the probe may have a relatively small insertion diameter (e.g. ≤6 mm or ≤18 Fr). As a result, placing a transnasal TEO probe will resemble the process used on commercial nasogastric tubes and therefore can be carried out by a minimally trained person without sedating the patient.

The disclosed TEO system utilizes photoacoustics, where light pulses at wavelengths differentially absorbed by oxygenated and de-oxygenated blood cause ultrasound to be generated in tissue, enabling the measurement of blood oxygenation for a blood vessel or a cardiac chamber of interest. For example, the device can obtain real-time sonographic images of cardiac structures. In addition, the device also can deliver light to and receive ultrasound from the pulmonary artery through the esophagus at a location determined through analyzing the sonographic images, making the measurement of mixed venous oxygenation much less invasive than the pulmonary catheters. Combining ultrasonic and photoacoustic measurement, it is feasible to derive a comprehensive set of hemodynamic information, such as heart rate, blood flow, blood pressure, preload, afterload, cardiac output, oxygen delivery, or oxygen consumption. Therefore, various embodiments of the TEO device may replace current highly invasive PACs with a low-risk comprehensive hemodynamic monitor, which may help save lives by enabling easy and reliable hemodynamic assessment near the point-of-care.

Disclosed herein are various embodiments of an apparatus and method for transesophageal echo-oximetry (TEO), in which a small probe is placed in the esophagus to simultaneously produce sonographic images and spectroscopic photoacoustic measurements from the heart. In various embodiments, the technology utilizes photoacoustics, in which light pulses at wavelengths differentially absorbed by oxygenated and de-oxygenated blood cause ultrasound to be generated in tissue, enabling the measurement of blood oxygenation for a blood vessel or a cardiac chamber of interest. For example, the device can deliver light to and receive ultrasound from the pulmonary artery through the esophagus, making the measurement of mixed venous oxygenation much less invasive than pulmonary catheters. In addition, embodiments of the disclosed device can simultaneously obtain real-time sonographic images of cardiac structures. Combining ultrasonic and photoacoustic measurements, it is feasible to derive a comprehensive set of hemodynamic information, such as heart rate, blood flow, blood pressure, preload, afterload, cardiac output, oxygen delivery, or oxygen consumption. Therefore, it is expected that TEO may replace the highly invasive PACs with a low-risk comprehensive hemodynamic monitor and help save lives by enabling easy and reliable hemodynamic assessment near the point-of-care.

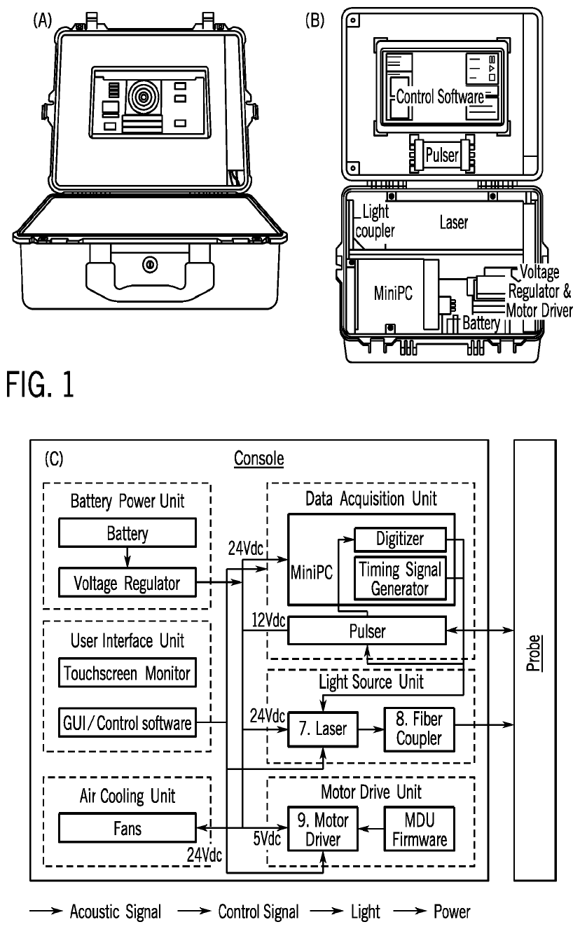

The disclosure describes several embodiments of a portable battery-powered portable TEO system and a miniature transnasal TEO probe which may be used at the point of care. Embodiments of the TEO system include a battery-powered device with a customized dual-wavelength light source suitable for in-vivo mixed venous oxygenation monitoring (FIGS. 1A and 1B). In various embodiments the device may fit into a portable suitcase measuring 62×49×22 cm³. As illustrated in the embodiment of FIG. 1C, this system includes six subunits: a light source unit, a battery power unit, a data acquisition unit, a motor drive unit, a ventilation unit, and a user interface unit.

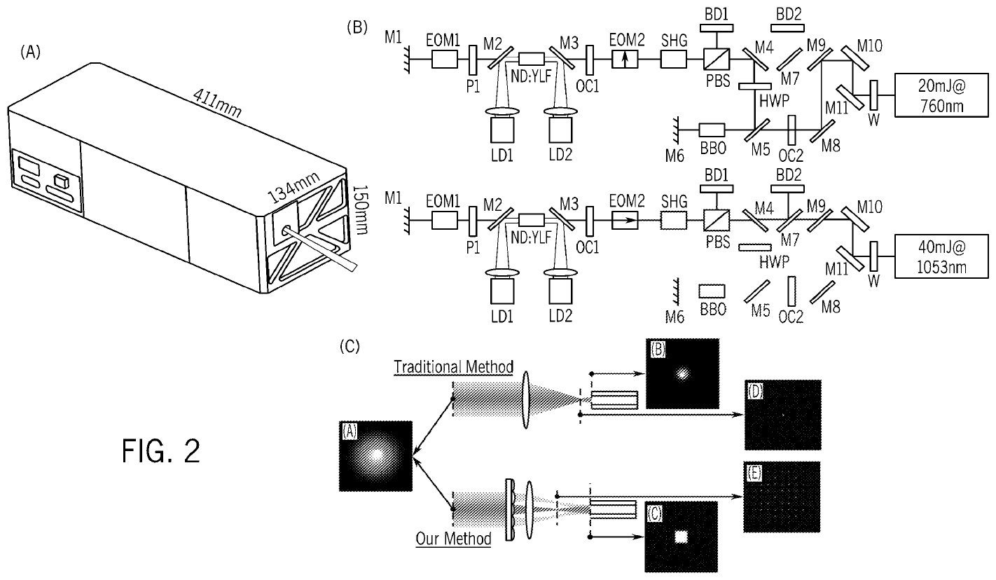

Reliable photoacoustic evaluation of blood oxygenation requires a light source capable of generating light pulses with a short pulse duration (1-100 ns) and sufficient energy (>10 mJ) and which can be switchable between two selected far-red or near-infrared wavelengths (e.g. between 600˜2500 nm), preferably near 760 nm (e.g. between 750 nm-770 nm) and near 1000 nm (e.g. between 900 nm-1100 nm), respectively. In various embodiments, each laser pulse group includes at least one pulse at each wavelength. The time interval between laser pulses in the same group may be 25 ms (range: 100 ns˜100 ms, with shorter time intervals being preferred to reduce or avoid measurement errors caused by tissue motion). The repetition rate of the pulse group may be 1 Hz (range: 0.1˜100 Hz). To make the final device portable, the light source in certain embodiments further needs to be compact in size, battery powered, and air cooled. In some embodiments, the light pulses are generated by an optical parametric oscillator (OPO) laser, where all optical and electronic components have been integrated in a compact housing measuring 41×13×15 cm³ (FIG. 2A). As illustrated in FIG. 2B, the laser features two resonant cavities. The first cavity (between M1 & OC1) was designed to produce light pulses at 1053 nm with a short duration of 3˜4 ns and high energy of over 90 mJ, through efficient energy extraction by a neodymium-doped yttrium lithium fluoride crystal (ND:YLF) from seed light provided by two high-power laser diodes (LD1 & LD2) as well as active Q-switching provided by a polarizer (P1) and an electro-optical modulator (EOM1) such as a Pockels cell. A second electro-optic modulator (EOM2) is used to shift the polarization of the 1053-nm light between vertical and horizontal orientation. When it is vertical, a second harmonic generator (SHG) converts these 1053-nm light pulses into green light at 526.5 nm. The second laser cavity (between M6 & OC2) use a nonlinear barium borate crystal (BBO) to further convert these green pulses into 760-nm light, which are directed by mirrors M9-M11 to the exit window (W) and which have energy of >20 mJ per pulse. When the polarization of the 1053-nm pulses is horizontal, they go unconverted directly to the exit window (W) and have energy of >40 mJ per pulse. The pulse duration at 760 nm and 1053 nm were measured to be 2.65 ns and 3.25 ns, respectively. In the external triggering mode, the laser emits two light pulses with any combination of the two wavelengths every second with an inter-pulse interval of 25 ms.

The OPO laser has two unique advantages: 1) It is pumped by energy-efficient laser diodes. Compared to flashlamp-pumped lasers used in typical photoacoustic devices, it has higher energy conversion efficiency, does not require using bulky liquid cooling systems, and generates little noise. Therefore, it enables system miniaturization with air cooling and quiet operation. 2) It implements a unique scheme to provide rapid pulse-to-pulse wavelength switching through electro-optic modulation. Compared to traditional methods using mechanical switching (e.g., using optical shutters), it not only is much faster but also causes minimal wear over long-time use. Therefore, it helps improve oxygenation evaluation accuracy through minimizing motion artifacts between photoacoustic measurements and extends the system's lifetime.

Remote delivery of mJ-level nanosecond light pulses usually requires using a bulky fiber bundle including many optical fibers to distribute light energy over a large input surface area to avoid damage to the fiber end faces. In some embodiments, an optical setup that can couple high-energy light pulses into a single optical fiber with high efficiency and robustness as shown in FIG. 2C. Traditional fiber coupling setups use one or more lenses to focus light into a single spot with high energy density on or before the fiber surface, therefore could cause breakdown of fiber or air there especially when hot spots exist in the incoming light beam (which occurs commonly with a OPO laser). Furthermore, because light at different wavelengths produces focal points with different sizes, it is also difficult to achieve high optical throughput consistently.

Embodiments of the disclosed procedures use a microlens array to split the incoming light beam into many sub-beams, then use a spherical lens to overlap them onto a small rectangular area on the fiber surface. In this way, we avoid laser-induced breakdown in fiber or air by distributing the energy. Heterogenous energy patterns in the incoming beam larger than the size of a single microlens element (0.5 mm in our setup) are homogenized to mitigate laser damage caused by hot spots (FIG. 2C, compare subpanels (B) and (C)).

Finally, the size of the light beam on the fiber surface is determined only by the parameter of the optical elements and remains independent of the light wavelength. The simulation showed that the lens-array setup reduced the peak optical energy density in the light path by more than 20 times, helping to avoid laser-induced breakdown on the fiber or in air by spreading the energy across multiple optical foci (FIG. 2C, compare subpanels (D) and (E)). Also, this method's coupling efficiency at 760 nm and 1053 nm differs by merely 11%, compared to 87% by the traditional method. Experimental tests further showed that the setup successfully delivered light from the OPO laser through a single 550-μm-core silica fiber with >70% throughput at both 760 nm and 1053 nm. No laser-induced damage to optical fiber was observed during use so far.

In various embodiments, the blood oxygenation may be evaluated once every second (e.g. at a rate of approximately 1 Hz), although in other embodiments the blood oxygenation may be evaluated at more or less frequent time intervals, for example ranging from 0.1 Hz to 100 Hz. During each measurement cycle, 38 ultrasonic images of the heart are obtained first, followed by two photoacoustic frames acquired at 1053 nm and 760 nm, respectively. For each image/frame, signal will be acquired at 250 distinct angular positions along a circle through rotating a reflector by a micromotor. The optimal position to measure photoacoustic signals from the pulmonary artery for mixed venous monitoring may be identified from the ultrasonic images First, the target of interest, e.g. the pulmonary artery, may be identified on the ultrasound images (e.g. at least one of Frame 1˜38), either by user annotation or by an automated imaging analysis algorithm (e.g. using machine learning). Then, light at the two selected wavelengths will be emitted at the angular position which crosses at about the middle of the target, e.g. in Frames 39 and 40, and two photoacoustic measurements will be taken, after which blood oxygenation of the target will be calculated. Based on such a procedure, laser firing may be automatically activated at those positions to acquire photoacoustic emission from the relevant positions. In the motor drive unit, a stepper motor controller was programmed to rotate the probe micromotor with high repeatability using 1/32 microstepping, to ensure that photoacoustic signals in the same cycle are acquired from the same angular position. In the data acquisition unit, a customized high-repetition radiofrequency pulser is used to generate ultrasound waves for ultrasonic imaging. A timing signal generation circuit generates TTL pulses to synchronize the firing of the laser and pulser, as well as data acquisition of ultrasonic and photoacoustic signals. The signals are then digitized by a high-speed digitizer and processed by a fanless mini-PC. Finally, control software has been developed in Labview, with computation-intensive data processing tasks implemented in C and included as dynamic-link libraries. Users will be able to operate the system, adjust settings, and observe results in real time through a touchscreen using a user-friendly graphical interface.

Console miniaturization is important for development of a portable device. To the best of the inventors' knowledge, the presently-disclosed battery-powered portable clinical-grade photoacoustic system is among the first of its kind. All system components are powered by a high-capacity 300-Wh rechargeable lithium-ion battery. On average, the system consumes 70˜90 W when actively monitoring oxygenation. It is estimated that a full charge could provide more than 3 hours of operation time. The console can also be plugged into a standard 110-V outlet to operate independently of the battery while the battery is recharging. Heat produced by system components may be directed to a central ventilation valley and then dissipated into the environment through ventilation fans installed at both ends of the valleys. Sheets of vibration-absorbing material (e.g. Sorbothane) were installed between the light source unit and the case to shield it from vibration during portable use.

### Transnasal TEO Probe

We further developed a small-diameter TEO probe, which can be introduced

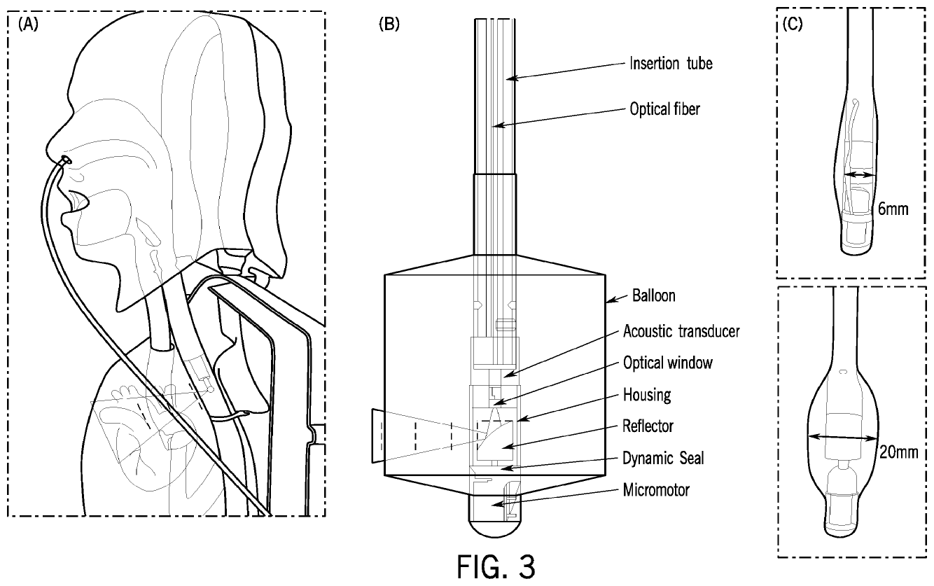

through the nose into the esophagus (FIG. 3A). The probe has a preferred insertion diameter of ≤6 mm (or 18 Fr). Placing a transnasal TEO probe will resemble the process used on commercial nasogastric tubes and therefore can be carried out by a minimally trained person without sedating the patient.

To obtain cross-sectional ultrasonic images of the heart, the acoustic detection and light excitation paths need to be circularly rotated. Two potential rotation schemes were explored: one using a distal micromotor, and the other using a proximal torque-transmission coil. Our studies showed that the micromotor-based solution enabled better measurement repeatability.

FIG. 3B illustrates an embodiment of the TEO probe featuring a distal micromotor. The probe generates and detects sound with a customized 6.5-MHz piezoceramic ring transducer (OD=5.5 mm, Hole diameter=1.5 mm, Active area=5.0 mm). Light from the OPO laser is first delivered to the distal end of the probe by a 550-μm double-clad silica fiber through the central hole in the transducer, then deflected by a rod reflector to illuminate tissue sideways (e.g. the reflector is at an angle of between about 40°-50°, preferably about 45°, relative to the long axis of the probe so that the tissue adjacent to the probe is illuminated). The reflector also reflects acoustic waves for ultrasonic imaging and photoacoustic measurements. The reflector is mounted on the micro-stepping-motor. The transducer, reflector, and micromotor may be aligned and assembled inside a CNC-machined plastic housing. For proper acoustic coupling, the housing may be made from materials that may have acoustic impedance close to that of oil, water or tissue, such as polymethyl methacrylate (PMMA), Polymer (TPX), or Rexolite. The housing may be also filled with an acoustic coupling fluid such as water, saline, silicone oil, corn oil, or mineral oil. To prevent contamination of the fiber and the micromotor by oil, an optical window may be used to seal the central hole in the transducer and a customized EPDM dynamic seal may be installed around the rotation shaft. The distal end of the probe may be further equipped with a polyurethane balloon which can be inflated by saline or water to a diameter of 20 mm-25 mm. In certain embodiments, a 5-lumen Pellethane® insertion tube has been custom extruded to house the optical fiber, the signal cables of the transducer, the electric wires of the motors, and the liquid channels for balloon inflation.

As shown in FIG. 3C, when the balloon is deflated, the fully assembled transnasal TEO probe has a maximal outer diameter on the distal end measuring at 6 mm, which is comparable in size to commercial nasogastric tubes (e.g., Salem Sump™ #8888265140, Cardinal). The probe has a small rigid length at the distal end of 25 mm, and a flexible shaft with a bending radius <35 mm and a bending angle >110°. Through testing on an adult nasogastric feeding training mannequin (Corman, Nasco), we found it could be readily inserted and retracted through a nostril into the esophagus (FIG. 3A). When placed into esophagus, the balloon can be inflated by water or saline up to 20 mm (Fig.3C) and achieves air-free contact with the esophageal to minimize sound transmission loss. Ultrasonic images will be obtained to locate the pulmonary artery and photoacoustic measurements will be activated to enable evaluation of mixed venous oxygenation.

### EXAMPLES

The following are non-limiting examples of embodiments of the disclosure.

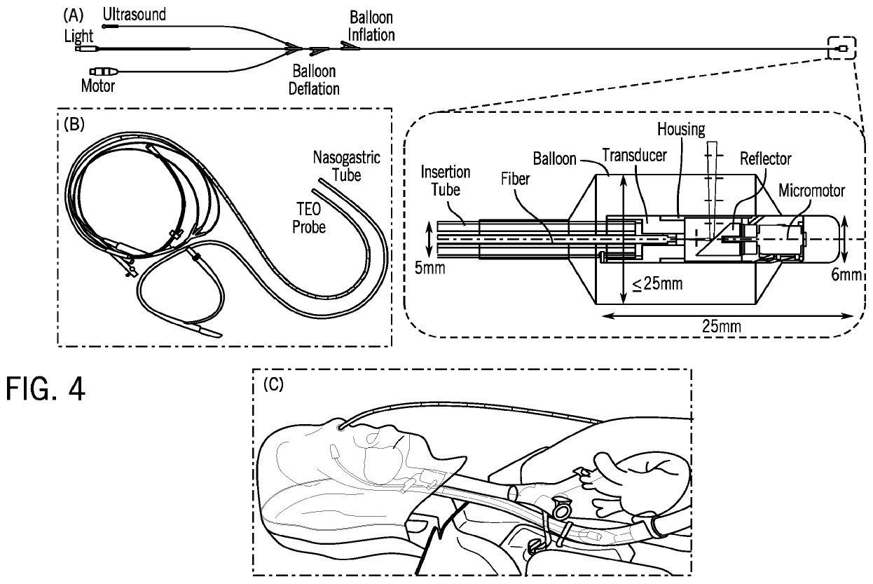

In one embodiment, a TEO probe featuring a miniature single-element ultrasound transducer and a rotating light/sound reflector was developed and used to perform tests (FIGS. 4A-4C). FIG. 4A shows engineering drawings of the probe depicting the proximal connectors and the internal configuration of the distal measurement tip (inset). FIG. 4B shows photos of the TEO probe alongside a commercial nasogastric tube. FIG. 4C shows transnasal placement of a TEO probe in a nasogastric feeding training mannequin. The inset of FIG. 4A shows a close-up view of the probe which provides exemplary dimensions of the TEO probe showing that the inflated balloon in this embodiment is approximately 25 mm in diameter, that the probe is about 25 mm in length, and that the insertion tube is approximately 5 mm in diameter while the body of the probe is approximately 6 mm in diameter. The cross-sectional view of the inset of FIG. 4A shows an embodiment of the ultrasound transducer which includes an opening through which the optical fiber is inserted. Also shown is a depiction of light (straight/expanding beam) and sound (groups of dots perpendicular to the light beam) reflecting off the reflector towards the side of the probe and into a tissue.

The reflector may be rotated to obtain cross-sectional ultrasonic images of the esophagus and surrounding large blood vessels/heart. In various embodiments, the reflector may be rotated using a distal micromotor or using a proximal metal torque-transmission coil. In the embodiment of FIGS. 4A-4C, a micromotor is used to rotate the reflector since studies showed that the micromotor-based solution provided better rotation repeatability needed to enable photoacoustic measurements from the target.

Before use, the probe is attached to the console by connecting a customized fiberoptic connector that transmits light to the tissue, a SMA connector for sending and receiving ultrasound signals, and an XLR connector for transmitting the signal that drives micromotor rotation. The distal measurement tip includes an ultrasound transducer, an optical fiber, a micromotor, a reflector, a housing, a balloon, and a flexible insertion tube. The 6.5-MHz ring-shaped piezoceramic transducer generates and detects ultrasound. The 550-μm double-clad silica fiber transmits light from the OPO laser to the tissue. The dielectric glass rod reflector re-directs both light and ultrasound at a 90-degree angle. The stepper micromotor rotates the reflector with good repeatability. The precision-machined optically and acoustically transparent acrylic housing helps align the micromotor, the reflector, and the transducer. It may also be filled with a fluid such as silicone or corn oil for better sound transmission. The optical fiber, the signal cables of the transducer, the electric wires of the motors, and the liquid channels for balloon inflation and deflation run through the custom-extruded 5-lumen Pellethane insertion tube. The polyurethane balloon encloses the entire distal end.

After the probe is introduced into the esophagus, the balloon may be inflated by water or saline to achieve air-free contact with the esophageal wall to minimize sound transmission loss. For identifying the target measurement location, the transducer sends a high-frequency sound wave, which bounces off the reflector towards the tissue. The sound wave partially reflected back from the tissue travels back to the transducer. The transducer converts the sound wave into electrical signals and sends them to the console. The console digitizes the signal and reconstructs cross-sectional sonographic images of tissue, from which the blood vessel of interest (e.g., a pulmonary artery or an aorta) can be identified. For the photoacoustic measurement, when the rotating reflector reaches the desired angle, the OPO laser generates a light pulse. The reflector redirects light out of the fiber to illuminate the targeted blood vessel. The generated photoacoustic signal is picked up by the transducer and processed by the console to determine the blood oxygenation in the targeted blood vessel.

FIG. 4B compares the TEO probe with a commercial nasogastric tube (Salem Sump™ #8888265140, Cardinal). When the balloon is deflated, the TEO probe has a maximal outer diameter measuring 6 mm, comparable in size to the commercially available 18-Fr nasogastric tube. The probe also has a small distal rigid length of 25 mm, a bending radius <20 mm, and a bending angle >110°. The probe was tested on a commercial adult nasogastric feeding training mannequin (Corman, Nasco). Results showed that it can be easily inserted into and retracted from the esophagus through both nostrils (FIG. 4C).

### Validation of the TEO Device in Swine Model In Vivo

The TEO device was used to test blood oxygenation monitoring in swine in vivo to measure mixed venous oxygenation (SvO₂) from pulmonary arteries in pigs undergoing controlled bleeding. In this example, the device was validated by measuring the arterial oxygenation (SaO₂) from the aorta in adult-sized pigs (Yorkshire, 60˜100 Kg). SaO₂ was modulated by changing the fractional oxygen in inspired gas (FiO₂). The aorta in pigs is located at a similar anatomical position to the pulmonary artery in humans; due to anatomical differences, the pulmonary artery in pigs is not close enough to the esophagus to perform measurements using the TEO probe and so the aorta was chosen instead for blood oxygenation testing purposes. For reference, TEO measurements were compared with the results from arterial blood gas analysis obtained by a commercial CO-oximeter (STAT PROFILE Prime+, Nova Biomedical). Arterial blood was withdrawn synchronously with the TEO measurements. By modulating FiO₂, a larger SaO₂ variation was produced in a gradual and controlled manner. It allowed us to validate the device's performance at lower blood oxygen levels, which would be seen with SvO₂ during hemorrhagic shock.

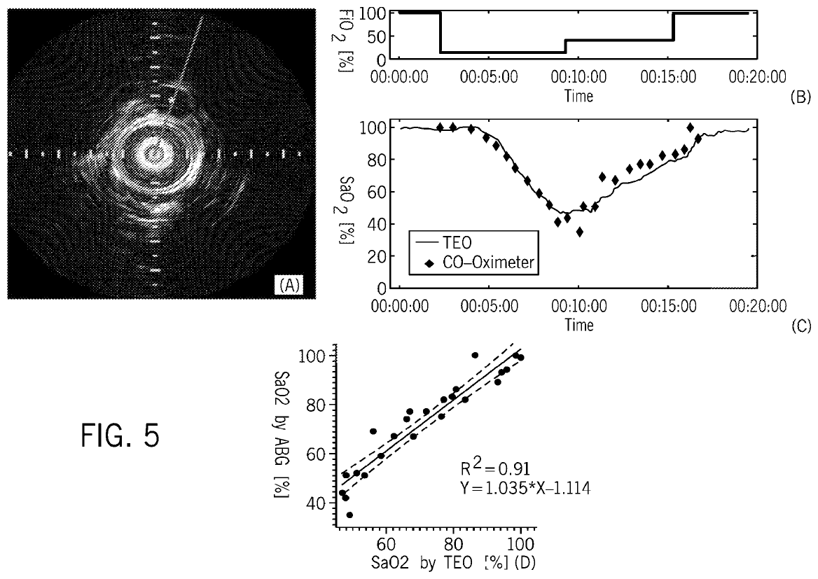

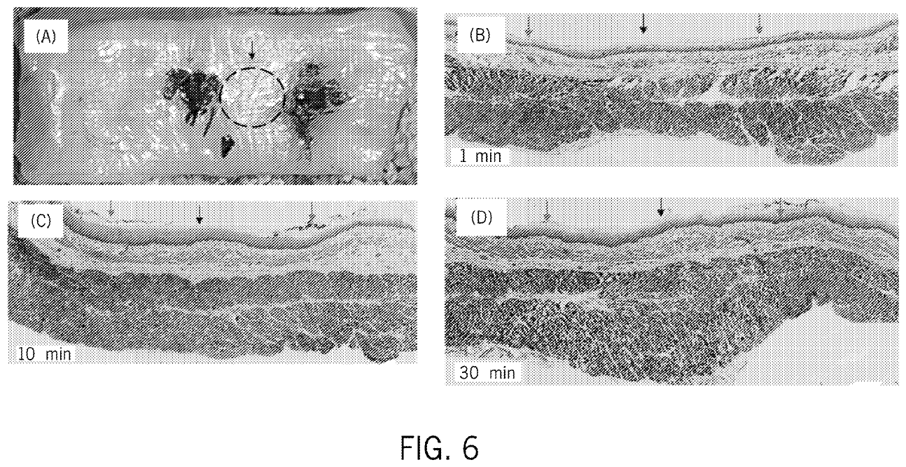

Results from the swine study showing the modulation of FiO₂can be seen in FIGS. 5A-5D. As shown in FIG. 5A, it was possible to easily identify the targeted blood vessel, i.e. the aorta, using the ultrasound images obtained by the TEO device (FIG. 5A, aorta is dark space having a yellow asterisk therein). Photoacoustic measurements were taken at the angular position marked by the red line (FIG. 6A). FIG. 5B shows how SaO₂ changed when we modulated FiO₂ by adjusting the ratio of O₂ and N₂ in the gas breathed by the animal. As expected, SaO₂ dropped from 100% to below 50% within 7 minutes after FiO₂ decreased from 100% to 15% and recovered when FiO₂ increased (FIGS. 5B-5C). We found that the blood SaO₂ measurements by TEO (FIG. 5C, green line) correlated well with the blood gas SaO₂ readings from the blood samples taken at the same time (FIG. 5C, red diamonds) with R²=0.91 (FIG. 5D).

### Validation That the TEO Light Exposure is Safe For Esophageal Tissue

Laser safety guidelines (ANSI Z136.1-2014) only provide maximum permissible exposure (MPE) for skin and eyes. The skin MPE is often used to guide the safe exposure limits to another opaque tissue type. Calculations identified that the light exposure used in TEO was below the Skin MPE (99% @ 760 nm and 51% @ 1053 nm under the single-shot criteria; 10% @ 760 nm and 5% @ 1053 nm under the average-power criteria).

TEO light exposure was further studied to determine if it would induce damage to esophageal tissue through either architectural changes or loss of cellular viability. Fresh swine esophagus was harvested immediately after sacrifice and immersed in CMRL cell culture media at 37° C. Then, a region on the luminal surface of the esophagus was exposed to the TEO light for 1, 10, and 30 minutes. To guide histological cutting, two registration sites flanking the exposure site were marked with red tissue ink (FIG. 6A). The tissue was trimmed close to the registration marks, mounted in Optimal Cutting Temperature media and subsequently frozen. Multiple histology levels (n=14) were cut starting at the edge of the red ink and sectioned through the spot at 400 μm intervals. The tissue slides were stained with nitro tetrazolium blue chloride (NBTC). Histochemical staining with NBTC is a well-established method of assessing tissue viability with higher sensitivity and specificity than that of standard histological stains. Viable tissue will stain blue, while non-viable thermally damaged tissue will not stain. All slides were read by an experienced pathologist. Tissue injury was assessed by inspection of the surface epithelium and underlying histological layers for apparent physical damage. Cellular injury was evaluated by noting discontinuity in the color of the stain from dark blue to clear over the exposed region.

Representative histology from full-thickness esophageal specimens exposed to 1, 10, and 30 minutes are presented in FIGS. 6B-6D, respectively. All slides were well stained in sites that were not been exposed to the TEO laser (FIGS. 6B-6D, red arrows), showing that the tissue is viable and NBTC stain is working. No lightening of the NBTC stain, which would have indicated thermal damage, was observed over the exposure sites (FIGS. 6B-6D, black arrows), demonstrating that TEO light exposure is safe for esophageal tissue.

### Computer and Optical Systems

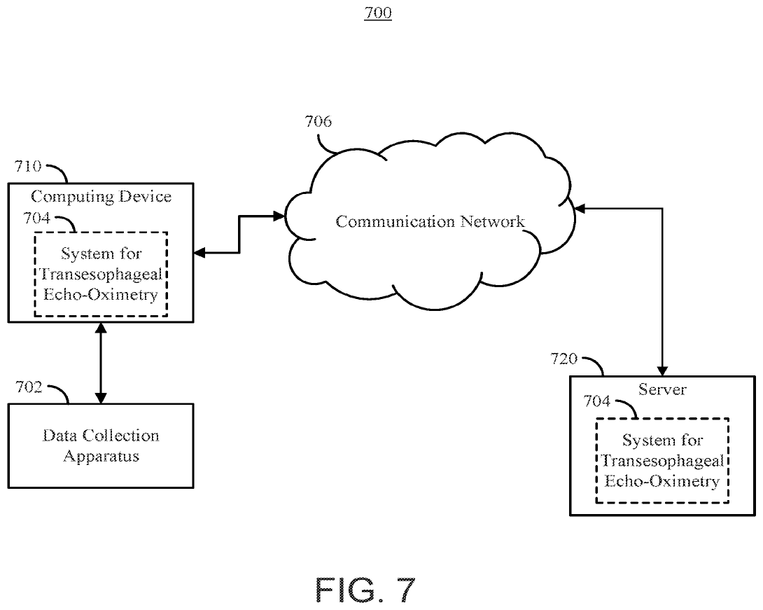

Turning to FIG. 7, an example 700 of a system (e.g. a data collection and processing system) for transesophageal echo-oximetry is shown in accordance with some embodiments of the disclosed subject matter. In some embodiments, a computing device 710 can execute at least a portion of a system for transesophageal echo-oximetry 704 and provide control signals to a data collection apparatus 702, e.g. such as the disclosed TEO probe. Additionally or alternatively, in some embodiments, computing device 710 can communicate information regarding the control signals to or from a server 720 over a communication network 706, which can execute at least a portion of system for transesophageal echo-oximetry 704. In some such embodiments, server 720 can return information to computing device 710 (and/or any other suitable computing device) relating to the control signals for system for transesophageal echo-oximetry 704. This information may be transmitted and/or presented to a user (e.g. a researcher, an operator, a clinician, etc.) and/or may be stored (e.g. as part of a research database or a medical record associated with a subject).

In some embodiments, computing device 710 and/or server 720 can be any suitable computing device or combination of devices, such as a desktop computer, a laptop computer, a smartphone, a tablet computer, a wearable computer, a server computer, a virtual machine being executed by a physical computing device, etc. As described herein, system for transesophageal echo-oximetry 704 can present information about the control signals to a user (e.g., researcher and/or physician).

In some embodiments, communication network 706 can be any suitable communication network or combination of communication networks. For example, communication network 706 can include a Wi-Fi network (which can include one or more wireless routers, one or more switches, etc.), a peer-to-peer network (e.g., a Bluetooth network), a cellular network (e.g., a 4G network, a 5G network, etc., complying with any suitable standard, such as CDMA, GSM, LTE, LTE Advanced, WiMAX, etc.), a wired network, etc. In some embodiments, communication network 706 can be a local area network, a wide area network, a public network (e.g., the Internet), a private or semi-private network (e.g., a corporate or university intranet), any other suitable type of network, or any suitable combination of networks. Communications links shown in FIG. 7 can each be any suitable communications link or combination of communications links, such as wired links, fiber optic links, Wi-Fi links, Bluetooth links, cellular links, etc.

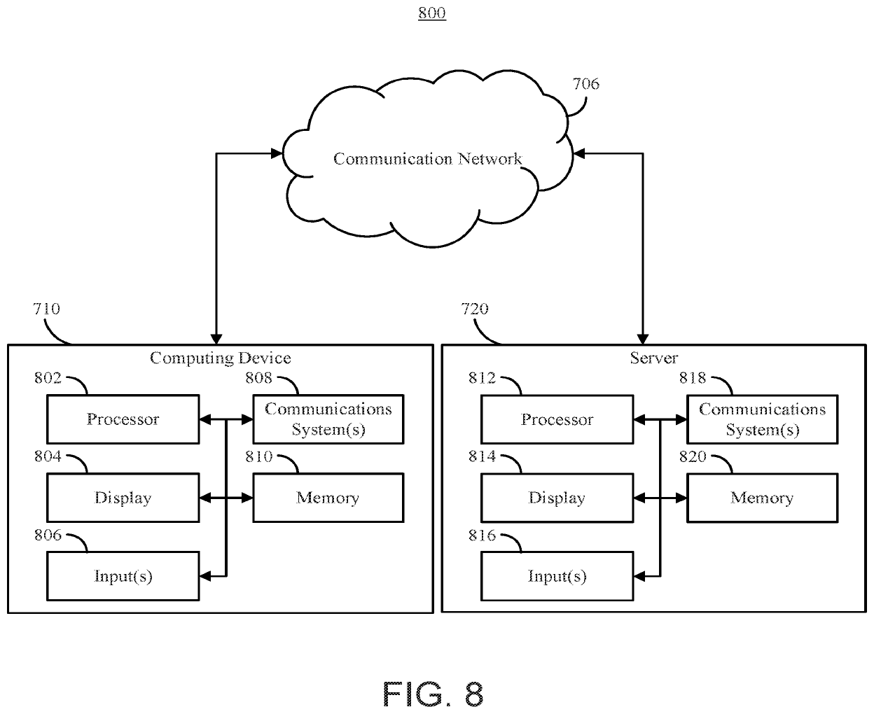

FIG. 8 shows an example 800 of hardware that can be used to implement computing device 710 and server 720 in accordance with some embodiments of the disclosed subject matter. As shown in FIG. 8, in some embodiments, computing device 710 can include a processor 802, a display 804, one or more inputs 806, one or more communication systems 808, and/or memory 810. In some embodiments, processor 802 can be any suitable hardware processor or combination of processors, such as a central processing unit, a graphics processing unit, etc. In some embodiments, display 804 can include any suitable display devices, such as a computer monitor, a touchscreen, a television, etc. In some embodiments, inputs 806 can include any suitable input devices and/or sensors that can be used to receive user input, such as a keyboard, a mouse, a touchscreen, a microphone, etc.

In some embodiments, communications systems 808 can include any suitable hardware, firmware, and/or software for communicating information over communication network 706 and/or any other suitable communication networks. For example, communications systems 808 can include one or more transceivers, one or more communication chips and/or chip sets, etc. In a more particular example, communications systems 808 can include hardware, firmware and/or software that can be used to establish a Wi-Fi connection, a Bluetooth connection, a cellular connection, an Ethernet connection, etc.

In some embodiments, memory 810 can include any suitable storage device or devices that can be used to store instructions, values, etc., that can be used, for example, by processor 802 to present content using display 804, to communicate with server 720 via communications system(s) 808, etc. Memory 810 can include any suitable volatile memory, non-volatile memory, storage, or any suitable combination thereof. For example, memory 810 can include RAM, ROM, EEPROM, one or more flash drives, one or more hard disks, one or more solid state drives, one or more optical drives, etc. In some embodiments, memory 810 can have encoded thereon a computer program for controlling operation of computing device 710. In such embodiments, processor 802 can execute at least a portion of the computer program to present content (e.g., images, user interfaces, graphics, tables, etc.), receive content from server 720, transmit information to server 720, etc.

In some embodiments, server 720 can include a processor 812, a display 814, one or more inputs 816, one or more communications systems 818, and/or memory 820. In some embodiments, processor 812 can be any suitable hardware processor or combination of processors, such as a central processing unit, a graphics processing unit, etc. In some embodiments, display 814 can include any suitable display devices, such as a computer monitor, a touchscreen, a television, etc. In some embodiments, inputs 816 can include any suitable input devices and/or sensors that can be used to receive user input, such as a keyboard, a mouse, a touchscreen, a microphone, etc.

In some embodiments, communications systems 818 can include any suitable hardware, firmware, and/or software for communicating information over communication network 706 and/or any other suitable communication networks. For example, communications systems 818 can include one or more transceivers, one or more communication chips and/or chip sets, etc. In a more particular example, communications systems 818 can include hardware, firmware and/or software that can be used to establish a Wi-Fi connection, a Bluetooth connection, a cellular connection, an Ethernet connection, etc.

In some embodiments, memory 820 can include any suitable storage device or devices that can be used to store instructions, values, etc., that can be used, for example, by processor 812 to present content using display 814, to communicate with one or more computing devices 710, etc. Memory 820 can include any suitable volatile memory, non-volatile memory, storage, or any suitable combination thereof. For example, memory 820 can include RAM, ROM, EEPROM, one or more flash drives, one or more hard disks, one or more solid state drives, one or more optical drives, etc. In some embodiments, memory 820 can have encoded thereon a server program for controlling operation of server 720. In such embodiments, processor 812 can execute at least a portion of the server program to transmit information and/or content (e.g., results of a tissue identification and/or classification, a user interface, etc.) to one or more computing devices 710, receive information and/or content from one or more computing devices 710, receive instructions from one or more devices (e.g., a personal computer, a laptop computer, a tablet computer, a smartphone, etc.), etc.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be noted that, as used herein, the term mechanism can encompass hardware, software, firmware, or any suitable combination thereof.

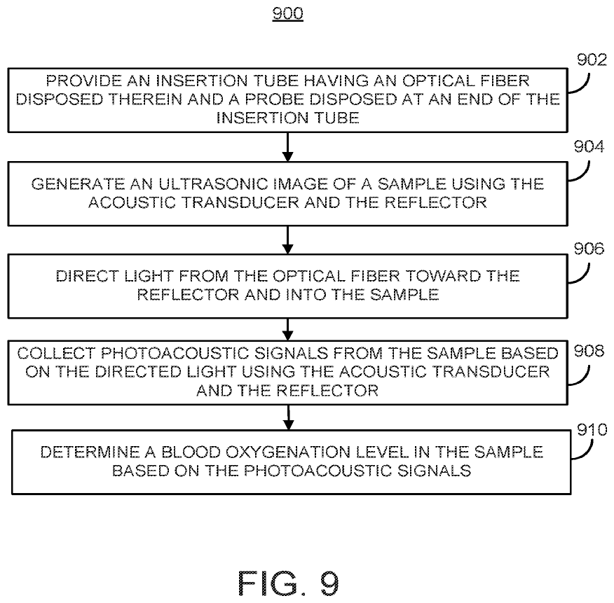

FIG. 9 shows an example 900 of a process for transesophageal echo-oximetry in accordance with some embodiments of the disclosed subject matter. As shown in FIG. 9, at 902, process 900 can provide an insertion tube having an optical fiber disposed therein and a probe disposed at an end of the insertion tube, where the probe may include an acoustic transducer, and a reflector aligned with the acoustic transducer and the optical fiber. At 904, process 900 can generate, using a controller in communication with the acoustic transducer, an ultrasonic image of a sample using the acoustic transducer and the reflector. At 906, process 900 can direct, using the controller, light from the optical fiber toward the reflector and into the sample. At 908, process 900 can collect, using the controller, photoacoustic signals from the sample based on the directed light using the acoustic transducer and the reflector. Finally, at 910, process 900 can determine, using the controller, a blood oxygenation level in the sample based on the photoacoustic signals.

It should be understood that the above described steps of the process of FIG. 9 can be executed or performed in any order or sequence not limited to the order and sequence shown and described in the figures. Also, some of the above steps of the processes of FIG. 9 can be executed or performed substantially simultaneously where appropriate or in parallel to reduce latency and processing times.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. An apparatus for transesophageal echo-oximetry, comprising:
an insertion tube having an optical fiber disposed therein;
a probe disposed at an end of the insertion tube, the probe comprising:
an acoustic transducer, and
a reflector aligned with the acoustic transducer and the optical fiber;
a controller in communication with the acoustic transducer, the controller configured to:
generate an ultrasonic image of a sample using the acoustic transducer and the reflector,
direct light from the optical fiber toward the reflector and into the sample,
collect photoacoustic signals from the sample based on the directed light using the acoustic transducer and the reflector, and
determine a blood oxygenation level in the sample based on the photoacoustic signals.

2. The apparatus of claim 1, wherein the probe further comprises a micromotor coupled to the reflector and in communication with the controller, and
wherein the controller is further configured to:
rotate the reflector using the micromotor,
generate the ultrasonic image of the sample using the acoustic transducer and the rotating reflector,
direct light from the optical fiber toward the rotating reflector and into the sample,
collect photoacoustic signals from the sample based on the directed light using the acoustic transducer and the rotating reflector, and
determine the blood oxygenation level in the sample based on the photoacoustic signals collected using the rotating reflector.

3. The apparatus of claim 1, further comprising a pulsed light source coupled to the optical fiber and in communication with the controller.

4. The apparatus of claim 3, wherein the pulsed light source emits light pulses having a pulse duration of at least 1 ns and no more than 100 ns.

5. The apparatus of claim 3, wherein the pulsed light source emits light pulses comprising near-infrared light.

6. The apparatus of claim 5, wherein the pulsed light source is switchable between two different wavelengths of light.

7. The apparatus of claim 6, wherein the pulsed light source emits light pulses comprising a wavelength in a range of at least one of 750 nm to 770 nm or 900 nm to 1100 nm.

8. The apparatus of claim 7, wherein the pulsed light source emits light pulses comprising a wavelength of at least one of 760 nm or 1053 nm.

9. The apparatus of claim 3, wherein the pulsed light source emits light pulses having an energy of greater than 10 mJ.

10. The apparatus of claim 3, wherein the pulsed light source is air cooled.

11. The apparatus of claim 3, wherein the pulsed light source includes a power supply comprising a battery.

12. The apparatus of claim 3, wherein the pulsed light source is coupled to the optical fiber using a microlens array and a spherical lens to project an output of the pulsed light source onto an end of the optical fiber.

13. The apparatus of claim 3, wherein the pulsed light source comprises:
a first resonant cavity powered by a pair of laser diodes;
a second resonant cavity in optical communication with the first optical cavity;
an electro-optic modulator; and
an output configured to emit light pulses,
wherein the electro-optic modulator has a first position which transmits light from the first resonant cavity to the output,
wherein the electro-optic modulator has a second position which transmits light from the first resonant cavity to the second resonant cavity,
wherein the light pulses emitted from the output comprises a first wavelength from the first resonant cavity when the electro-optic modulator is in the first position, and
wherein the light pulses emitted from the output comprises a second wavelength different from the first wavelength from the second resonant cavity when the electro-optic modulator is in the second position.

14. The apparatus of claim 1, wherein the optical fiber extends through an opening in the acoustic transducer.

15. The apparatus of claim 1, wherein the probe comprises a housing having the reflector and the acoustic transducer disposed therein, and
wherein the housing includes an acoustic coupling fluid disposed therein.

16. The apparatus of claim 1, wherein the controller, when determining a blood oxygenation level in the sample based on the photoacoustic signals, is further configured to:
determine a blood oxygenation level in the sample based on the photoacoustic signals once per second.

17. The apparatus of claim 1, wherein the controller, when determining a blood oxygenation level in the sample based on the photoacoustic signals, is further configured to:
determine at least one of heart rate, blood flow, blood pressure, preload, afterload, cardiac output, oxygen delivery, or oxygen consumption in the sample based on the photoacoustic signals and the ultrasonic image of the sample.

18. The apparatus of claim 1, wherein a diameter of the probe is 6 mm or less.

19. The apparatus of claim 18, wherein the probe is configured to be delivered through a transnasal tube.

20. The apparatus of claim 1, wherein the probe is disposed within a balloon, and
wherein the balloon is inflated using a fluid.

21. The apparatus of claim 1, further comprising a portable power supply,
wherein the apparatus is configured to be stored in a portable case.

22-41. (canceled)
